(19) United States
(12) Patent Application Publication
(10) Pub. No.: US 2006/0223709 A1
(43) Pub. Date: Oct. 5, 2006

(54) Substituted pyrazolyloxyphenyl derivatives as herbicides

(75) Inventors: Hendrik Helmke, Liederbach (DE); Michael Gerhard Hoffmann, Florsheim (DE); Lothar Willms, Hofheim (DE); Thomas Auler, Leichlingen (DE); Heinz Kehne, Hofheim (DE); Martin Hills, Idstein (DE); Dieter Feucht, Eschborn (DE)

Correspondence Address: CONNOLLY BOVE LODGE & HUTZ, LLP, P O BOX 2207, WILMINGTON, DE, 19899, US

(73) Assignee: Bayer CropScience GmbH, Frankfurt am Main (DE)

(21) Appl. No.: 11/392,013

(22) Filed: Mar. 29, 2006

(30) Foreign Application Priority Data
Mar. 31, 2005 (DE) 10 2005 014 638.4

(51) Int. Cl. A01N 43/48; A01N 43/56; C07D 409/02; C07D 403/02

(52) U.S. Cl. 504/253; 504/282; 546/276.1; 548/364.1; 548/366.1

(57) ABSTRACT

What is described are pyrazolyloxyphenyl derivatives of the formula (I) and their use as herbicides. 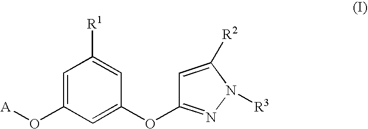 In this formula (I), R1, R2 and R3 denote various radicals, and A is a cyclic radical from the group consisting of phenyl, pyridyl, thienyl and pyrazolyl.

The invention relates to the technical field of the herbicides, in particular that of the herbicides for the selective control of broad-leaved weeds and weed grasses in crops of useful plants.

From U.S. Pat. No. 5,698,495, U.S. Pat. No. 5,786,392 and WO 9718196, it is already known that certain pyrazolyloxyphenyl derivatives have herbicidal properties. WO 2003/051846 likewise describes pyrazolyloxyphenyl derivatives carrying a substituted radical from the group consisting of phenyl, pyridyl, pyrazolyl and thienyl, which radical is attached via an oxygen atom.

However, the herbicidal activity of the compounds known from these publications is frequently insufficient. It is therefore an object of the present invention to provide herbicidally active compounds having herbicidal properties which are better than those of the compounds disclosed in the prior art.

It has now been found that pyrazolyloxyphenyl derivatives substituted by selected radicals are particularly suitable for use as herbicides. Part of the subject matter of the present invention are therefore compounds of the formula (I) and salts thereof 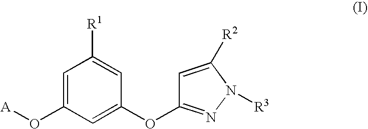
in which the substituents and indices are as defined below:
R¹ is hydrogen, bromine, chlorine, fluorine, iodine, or methylthio;
R² is trifluoromethyl, difluoromethyl or chlorodifluoromethyl;
R³ is methyl or ethyl;
A is a radical from the group consisting of the radicals A1 to A4 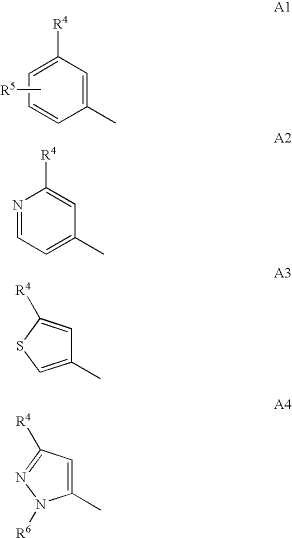
R⁴ is fluoromethyl, difluoromethyl, trifluoromethyl, fluoromethoxy, difluoromethoxy, trifluoromethoxy, chlorine or cyano;
R⁵ is hydrogen, (C₁-C₈)-alkyl, bromine, chlorine, fluorine, iodine or cyano, and
R⁶ is (C₁-C₈)-alkyl.

Depending on the nature of the substituents, the compounds of the formula (I) are capable of forming an adduct with an acid, for example hydrochloric acid. The acid adducts formed in this manner, such as hydrochlorides, also form part of the subject matter of the invention.

In formula (I) and all subsequent formulae, alkyl radicals with more than two carbon atoms can be straight-chain or branched. Alkyl radicals are, for example, methyl, ethyl, n- or i-propyl, n-, i-, t- or 2-butyl, pentyls, hexyls, such as n-hexyl, i-hexyl and 1,3-dimethylbutyl.

Depending on the type and the linkage of the substituents, the compounds of the formula (I) can exist as stereoisomers. If, for example, one or more asymmetric carbon atoms are present, enantiomers and diastereomers may occur. Stereoisomers can be obtained from the mixtures resulting from the preparation by means of customary separation methods, for example by chromatographic separation methods. Likewise, stereoisomers may be prepared selectively by using stereoselective reactions employing optically active starting materials and/or auxiliaries. The invention also relates to all stereoisomers and their mixtures which are encompassed by the formula (I), but not defined specifically.

Preference is given to compounds of the formula (I) in which

R¹ is hydrogen, bromine, chlorine, fluorine, iodine or methylthio;

R² is trifluoromethyl or difluoromethyl;

R³ is methyl or ethyl;

A is a radical from the group consisting of the radicals A1 to A4;

R⁴ is difluoromethyl, trifluoromethyl, difluoromethoxy, trifluoromethoxy, chlorine or cyano;

R⁵ is hydrogen, fluorine or chorine;

R⁶ is methyl or ethyl.

Particular preference is given to compounds of the formula (I) in which

R¹ is hydrogen, bromine, chlorine, fluorine, iodine or methylthio;

R² is trifluoromethyl or difluoromethyl;

R³ is methyl or ethyl;

A is a radical from the group consisting of the radicals A1 to A4;

R⁴ is difluoromethyl, trifluoromethyl, difluoromethoxy, trifluoromethoxy, chlorine or cyano;

R⁵ is hydrogen or fluorine;

R⁶ is methyl.

Very particular preference is given to compounds of the formula (I) in which
R¹ is hydrogen, bromine, chlorine, fluorine or iodine, preferably bromine, chlorine, fluorine or iodine;
R² is trifluoromethyl or difluoromethyl;
R³ is methyl or ethyl;
A is the radical A1;
R⁴ is difluoromethyl, trifluoromethyl, difluoromethoxy, trifluoromethoxy, chlorine or cyano;
R⁵ is hydrogen or fluorine.

Very particular preference is likewise given compounds of the formula (I) in which
R¹ is hydrogen, bromine, chlorine, fluorine or iodine, preferably bromine, chlorine, fluorine or iodine;
R² is trifluoromethyl or difluoromethyl;
R³ is methyl or ethyl;
A is the radical A2;
R⁴ is difluoromethyl, trifluoromethyl, difluoromethoxy, trifluoromethoxy, chlorine or cyano.

In addition, very particular preference is given to compounds of the formula (I) in which
R¹ is hydrogen, bromine, chlorine, fluorine or iodine, preferably bromine, chlorine, fluorine or iodine;
R² is trifluoromethyl or difluoromethyl;
R³ is methyl or ethyl;
A is the radical A3;
R⁴ is difluoromethyl, trifluoromethyl, difluoromethoxy, trifluoromethoxy, chlorine or cyano.

Very particular preference is likewise given to compounds of the formula (I) in which
R¹ is hydrogen, bromine, chlorine, fluorine or iodine, preferably bromine, chlorine, fluorine or iodine;
R² is trifluoromethyl or difluoromethyl;
R³ is methyl or ethyl;
A is the radical A4;
R⁴ is difluoromethyl, trifluoromethyl, difluoromethoxy, trifluoromethoxy, chlorine or cyano;
R⁶ represents methyl.

In all of the formulae given hereinbelow, the substituents and symbols have the same meaning as described under formula (I), unless defined otherwise.

Compounds of the formula (I) according to the invention can be prepared, for example, by the process shown in scheme 1. Here, in a first step, the compound of the formula (II) is, with base induction, reacted with a compound A-OH and then, in a second step, also with base induction, reacted with a compound (IIIa), to give compounds of the formula (Ia) according to the invention in which R¹ is nitro. In the compounds of the formulae (II) and (IIa), LG is in each case a leaving group, such as chlorine, fluorine or pseudohalogen. These reactions are known to the person skilled in the art.

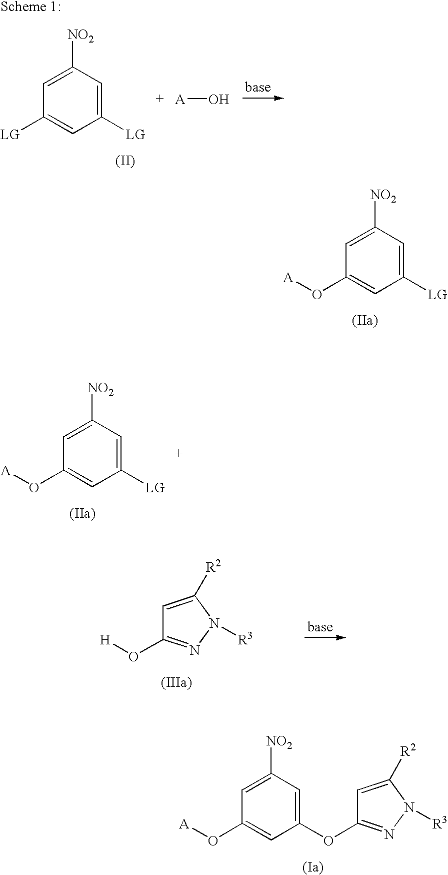

If required, the two reaction steps mentioned above can also be carried out in reverse order.

According to scheme 2, compounds of the formula (Ia) according to the invention can be converted into compounds of the formula (Ib) according to the invention in which R¹ is amino. These reactions are known to the person skilled in the art, for example from R. L. Augustine “Catalytic Hydrogenation” Marcel Dekker, New York 1965, Chpt 5, and P. N. Rylander “Hydrogenation Methods” Academic Press, New York 1985, Chpt 8.

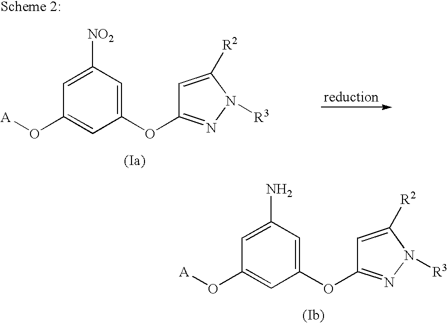

According to scheme 3, compounds of the formula (Ic) according to the invention in which R^{1a} is hydrogen, bromine, chlorine, fluorine, iodine or thiomethyl can be prepared by diazotization and subsequent functionalization from the compounds (Ib). The diazotization of the aniline derivative (III) and functionalization of the diazonium salts (boiling down and reduction, Schiemann reaction, Balz-Schiemann reaction, Sandmeyer reactions) are known to the person skilled in the art and can be carried out by known methods, see, for example,
a) F. A. Carey, R. J. Sundberg, Organische Chemie (Deutsche Ausgabe) [Organic Chemistry (German edition)] VCH Verlagsgesellschaft, Weinheim 1995, Chpt 24.2.1 and literature cited therein.
b) Organikum, VEB Deutscher Verlag der Wissenschaften, Berlin 1988, Chpt D.8.2.1, D.8.3.1, D.8.3.2 and literature references given in D.8.6.
c) Schank K., Aromatic diazonium salts. Method. Chim. (1975), δ 159-203.
d) Yoneda, Norihiko; Fukuhara, Tsuyoshi. Preparation of fluoro aromatics. Diazotization, fluorodediazoniation of amino aromatics. Yuki Gosei Kagaku Kyokaishi (1989), 47(7), 619-28.
e) Nonhebel, Derek C. Copper-catalyzed single-electron oxidations and reductions. Special Publication—Chemical Society (1970), No. 24 409-37.

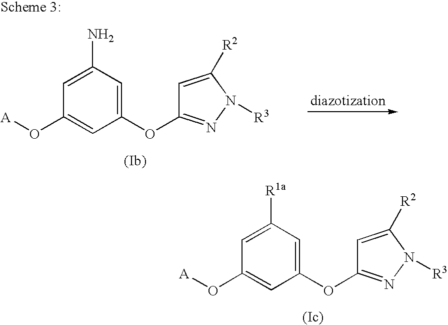

The compounds of the formula (I) according to the invention have an excellent herbicidal activity against a broad range of economically important monocotyledonous and dicotyledonous harmful plants. The active substances control perennial weeds equally well which produce shoots from rhizomes, root stocks or other perennial organs and which cannot be easily controlled. In this context, it generally does not matter whether the substances are applied before sowing, pre-emergence or post-emergence. Some representatives of the monocotyledonous and dicotyledonous weed flora which can be controlled by the compounds according to the invention may be mentioned individually as examples, but this is not to be taken to mean a restriction to certain species. The monocotyledonous weed species which are controlled well are, for example, Avena, Lolium, Alopecurus, Phalaris, Echinochloa, Digitaria, Setaria and Cyperus species from the annual group, and Agropyron, Cynodon, Imperata and Sorghum or else perennial Cyperus species amongst the perennial species. In the case of dicotyledonous weed species, the spectrum of action extends to species such as, for example, Galium, Viola, Veronica, Lamium, Stellaria, Amaranthus, Sinapis, Ipomoea, Sida, Matricaria and Abutilon from the annual group, and Convolvulus, Cirsium, Rumex and Artemisia among the perennial weeds. Harmful plants which are found under the specific culture conditions of rice, such as, for example, Echinochloa, Sagittaria, Alisma, Eleocharis, Scirpus and Cyperus are also controlled outstandingly well by the active substances according to the invention. If the compounds according to the invention are applied to the soil surface prior to germination, then either emergence of the weed seedlings is prevented completely, or the weeds grow until they have reached the cotyledon stage but growth then comes to a standstill and, after a period of three to four weeks, the plants eventually die completely. When the active substances are applied post-emergence to the green parts of the plants, growth also stops drastically very soon after the treatment, and the weeds remain at the growth stage of the time of application, or, after a certain period of time, they die completely so that competition by the weeds, which is detrimental for the crop plants, is thus eliminated at a very early stage and in a sustained manner. In particular, the compounds according to the invention have an outstanding action against Apera spica venti, Chenopodium album, Lamium purpureum, Polygonum convulvulus, Stellaria media, Veronica hederifolia, Veronica persica and Viola tricolor.

Although the compounds according to the invention have an outstanding herbicidal activity against monocotyledonous and dicotyledonous weeds, crop plants of economically important crops such as, for example, wheat, barley, rye, rice, corn, sugar beet, cotton and soybeans, only suffer negligible damage, if any. In particular, they are outstandingly well tolerated in cereals, such as wheat, barley and corn, in particular wheat. This is why the present compounds are highly suitable for the selective control of undesired vegetation in stands of agricultural useful plants or of ornamentals.

Owing to their herbicidal properties, the active substances can also be employed for controlling harmful plants in crops of known plants or genetically modified plants which are yet to be developed. As a rule, the transgenic plants are distinguished by particularly advantageous properties, for example by resistances to certain pesticides, especially certain herbicides, by resistances to plant diseases or causative organisms of plant diseases, such as certain insects or microorganisms such as fungi, bacteria or viruses. Other particular properties concern for example the harvested material with regard to quantity, quality, shelf life, composition and specific constituents. Thus, transgenic plants are known which have an increased starch content or whose starch quality has been modified, or those whose fatty acid composition in the harvested material is different.

The compounds of the formula (I) according to the invention or their salts are preferably employed in economically important transgenic crops of useful plants and ornamentals, for example cereals such as wheat, barley, rye, oats, millet, rice, cassava and corn, or else crops of sugar beet, cotton, soybeans, oilseed rape, potato, tomato, pea and other vegetables. The compounds of the formula (I) can preferably be employed as herbicides in crops of useful plants which are resistant, or have been genetically modified to be resistant, to the phytotoxic effects of the herbicides.

Conventional routes for the generation of novel plants which have modified properties compared with existing plants are, for example, traditional breeding methods and the generation of mutants. Alternatively, novel plants with modified properties can be generated with the aid of recombinant methods (see, for example, EP-A-0221044, EP-A-0131624). For example, several cases of the following have been described:
recombinant modifications of crop plants for the purposes of modifying the starch synthesized in the plants (for example WO 92/11376, WO 92/14827, WO 91/19806),
transgenic crop plants which exhibit resistances to certain herbicides of the glufosinate type (cf. eg. EP-A-0242236, EP-A-242246), glyphosate type (WO 92/00377) or of the sulfonylurea type (EP-A-0257993, U.S. Pat. No. 5,013,659)
transgenic crop plants, for example cotton, with the ability to produce Bacillus thuringiensis toxins (Bt toxins), which make the plants resistant to certain pests (EP-A-0142924, EP-A-0193259),
transgenic crop plants with a modified fatty acid composition (WO 91/13972),

A large number of techniques in molecular biology, with the aid of which novel transgenic plants with modified properties can be generated, are known in principle; see, for example, Sambrook et al., 1989, Molecular Cloning, A Laboratory Manual, 2^{nd} Ed., Cold Spring Harbor Laboratory Press, Cold Spring Harbor, N.Y.; or Winnacker “Gene und Klone” [Genes and Clones], VCH Weinheim 2^{nd} Edition 1996 or Christou, “Trends in Plant Science” 1 (1996) 423-431.

To carry out such recombinant manipulations, nucleic acid molecules can be introduced into plasmids which permit a mutagenesis or a sequence alteration by recombination of DNA sequences. With the aid of the abovementioned standard methods, it is possible, for example, to carry out base substitutions, to remove part sequences or to add natural or synthetic sequences. The fragments can be provided with adapters or linkers to link the DNA fragments to each other.

Plant cells with a reduced activity of a gene product can be obtained, for example, by expressing at least one corresponding antisense RNA, a sense RNA for achieving a cosuppression effect, or the expression of at least one suitably constructed ribozyme which specifically cleaves transcripts of the abovementioned gene product.

To this end, it is possible, on the one hand, to use DNA molecules which encompass all of the coding sequence of a gene product including any flanking sequences which may be present, but also DNA molecules which only encompass portions of the coding sequence, it being necessary for these portions to be so long as to cause an antisense effect in the cells. Another possibility is the use of DNA sequences which have a high degree of homology with the coding sequences of a gene product, but are not completely identical.

When expressing nucleic acid molecules in plants, the protein synthesized may be localized in any desired compartment of the plant cell. However, to achieve localization in a particular compartment, the coding region can, for example, be linked to DNA sequences which ensure localization in a particular compartment. Such sequences are known to the skilled worker (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give intact plants. In principle, the transgenic plants can be plants of any desired plant species, i.e. both monocotyledonous and dicotyledonous plants.

Thus, transgenic plants can be obtained which exhibit modified properties owing to the overexpression, suppression or inhibition of homologous (=natural) genes or gene sequences or expression of heterologous (=foreign) genes or gene sequences.

When using the active substances according to the invention in transgenic crops, effects are frequently observed in addition to the effects against harmful plants to be observed in other crops, which are specific for the application in the transgenic crop in question, for example a modified or specifically widened weed spectrum which can be controlled, modified application rates which may be employed for the application, preferably good combining ability with the herbicides to which the transgenic crop is resistant, and an effect on the growth and yield of the transgenic crop plants. The invention therefore also relates to the use of the compounds according to the invention as herbicides for controlling harmful plants in transgenic crop plants.

The substances according to the invention additionally have outstanding growth-regulatory properties in crop plants. They engage in the plants' metabolism in a regulatory fashion and can thus be employed for the targeted control of plant constituents and for facilitating harvesting, such as, for example, triggering desiccation and stunted growth. Moreover, they are also suitable for generally controlling and inhibiting undesired vegetative growth without destroying the plants in the process. Inhibiting the vegetative growth plays an important role in many monocotyledonous and dicotyledonous crops since lodging can be reduced, or prevented completely, hereby.

The compounds according to the invention can be employed in the form of wettable powders, emulsifiable concentrates, sprayable solutions, dusts or granules in the customary preparations. The invention therefore furthermore relates to herbicidal compositions comprising compounds of the formula (I). The compounds of the formula (I) can be formulated in various ways, depending on the prevailing biological and/or chemico-physical parameters. Examples of suitable formulations which are possible are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing products, granules for spreading and soil application, granules (GR) in the form of microgranules, spray granules, coated granules and adsorption granules, water-dispersible granules (WG), water-soluble granules (SG), ULV formulations, microcapsules and waxes. These individual formulation types are known in principle and are described, for example, in Winnacker-Küchler, “Chemische Technologie” [Chemical Engineering], Volume 7, C. Hauser Verlag Munich, 4th Ed. 1986, Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J., H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie”, Volume 7, C. Hauser Verlag Munich, 4th Ed. 1986.

Wettable powders are preparations which are uniformly dispersible in water and which, in addition to the active substance, also contain ionic and/or nonionic surfactants (wetters, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium 2,2′-dinaphthylmethane-6,6′-disulfonate, sodium lignosulfonate, sodium dibutylnaphthalenesulfonate or else sodium oleoylmethyltaurate, in addition to a diluent or inert substance. To prepare the wettable powders, the herbicidal active substances are ground finely, for example in customary equipment such as hammer mills, blowing mills and air-jet mills, and simultaneously or subsequently mixed with the formulation auxiliaries.

Emulsifiable concentrates are prepared by dissolving the active substance in an organic solvent, e.g. butanol, cyclohexanone, dimethylformamide, xylene or else higher-boiling aromatics or hydrocarbons or mixtures of the organic solvents with addition of one or more ionic and/or nonionic surfactants (emulsifiers). Examples of emulsifiers which can be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan esters such as, for example, sorbitan fatty acid esters or polyoxyethylene sorbitan esters such as, for example, polyoxyethylene sorbitan fatty acid esters.

Dusts are obtained by grinding the active substance with finely divided solid materials, for example talc, natural clays such as kaolin, bentonite and pyrophyllite, or diatomaceous earth.

Suspension concentrates can be water-based or oil-based. They can be prepared for example by wet-grinding by means of customary bead mills, if appropriate with addition of surfactants, as have already been mentioned for example above in the case of the other formulation types.

Emulsions, for example oil-in-water emulsions (EW), can be prepared for example by means of stirrers, colloid mills and/or static mixers using aqueous organic solvents and, if appropriate, surfactants as have already been mentioned for example above in the case of the other formulation types.

Granules can be prepared either by spraying the active substance onto adsorptive, granulated inert material or by applying active substance concentrates to the surface of carriers such as sand, kaolinites or granulated inert material with the aid of adhesives, for example polyvinyl alcohol, sodium polyacrylate or else mineral oils. Suitable active substances can also be granulated in the fashion which is conventional for the production of fertilizer granules, if desired as a mixture with fertilizers.

Water-dispersible granules are generally prepared by customary methods such as spray drying, fluidized-bed granulation, disk granulation, mixing with high-speed stirrers and extrusion without solid inert material.

To prepare disk granules, fluidized-bed granules, extruder granules and spray granules, see, for example methods in “Spray-Drying Handbook” 3rd ed. 1979, G. Goodwin Ltd., London; J. E. Browning, “Agglomeration”, Chemical and Engineering 1967, pages 147 et seq.; “Perry's Chemical Engineer's Handbook”, 5th Ed., McGraw-Hill, New York 1973, pp. 8-57.

For further details on the formulation of crop protection agents see, for example G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

As a rule, the agrochemical preparations comprise 0.1 to 99% by weight, in particular 0.1 to 95% by weight, of active substance of the formula (I). In wettable powders, the active substance concentration is, for example, approximately 10 to 90% by weight, the remainder to 100% by weight being composed of customary formulation constituents. In the case of emulsifiable concentrates, the active substance concentration can amount to approximately 1 to 90, preferably 5 to 80% by weight. Formulations in the form of dusts comprise 1 to 30% by weight of active substance, preferably in most cases 5 to 20% by weight of active substance, and sprayable solutions comprise approximately 0.05 to 80, preferably 2 to 50% by weight of active substance. In the case of water-dispersible granules, the active substance content depends partly on whether the active compound is in liquid or solid form and on the granulation auxiliaries, fillers and the like which are being used. In the case of the water-dispersible granules, for example, the active substance content is between 1 and 95% by weight, preferably between 10 and 80% by weight.

In addition, the active substance formulations mentioned comprise, if appropriate, the tackifiers, wetters, dispersants, emulsifiers, penetrants, preservatives, antifreeze agents, solvents, fillers, carriers, colorants, antifoams, evaporation inhibitors, and pH and viscosity regulators which are conventional in each case.

Based on these formulations, it is also possible to prepare combinations with other pesticidally active substances such as, for example, insecticides, acaricides, herbicides, fungicides, and with safeners, fertilizers and/or growth regulators, for example in the form of a readymix or a tank mix.

Active substances which can be employed in combination with the active substances according to the invention in mixed formulations or in the tank mix are, for example, known active substances as are described, for example, in Weed Research 26, 441-445 (1986) or “The Pesticide Manual”, 11th edition, The British Crop Protection Council and the Royal Soc. of Chemistry, 1997 and literature cited therein. Known herbicides which must be mentioned, and can be combined with the compounds of the formula (I), are, for example, the following active substances (note: the compounds are either designated by the common name according to the International Organization for Standardization (ISO) or using the chemical name, if appropriate together with a customary code number): acetochlor; acifluorfen; aclonifen; AKH 7088, i.e. [[[1-[5-[2-chloro-4-(trifluoromethyl)-phenoxy]-2-nitrophenyl]-2-methoxyethylidene]amino]oxy]acetic acid and its methyl ester; alachlor; alloxydim; ametryn; amidosulfuron; aminopyralid; amitrol; AMS, i.e. ammonium sulfamate; anilofos; asulam; atrazine; azimsulfurone (DPX-A8947); aziprotryn; barban; BAS 516H, i.e. 5-fluorine-2-phenyl-4H-3,1-benzoxazin-4-one; benazolin; benfluralin; benfuresate; bensulfuronmethyl; bensulide; bentazone; benzofenap; benzofluor; benzoylprop-ethyl; benzthiazuron; bialaphos; bifenox; bromacil; bromobutide; bromofenoxim; bromoxynil; bromuron; buminafos; busoxinone; butachlor; butamifos; butenachlor; buthidazole; butralin; butylate; cafenstrole (CH-900); carbetamide; cafentrazone (ICI-A0051); CDAA, i.e. 2-chloro-N,N-di-2-propenylacetamide; CDEC, i.e. 2-chloroallyl diethyldithiocarbamate; chlomethoxyfen; chloramben; chlorazifop-butyl, chlormesulon (ICI-A0051); chlorbromuron; chlorbufam; chlorfenac; chlorflurecol-methyl; chloridazon; chlorimuron ethyl; chlornitrofen; chlorotoluron; chloroxuron; chlorpropham; chlorsulfuron; chlorthal-dimethyl; chlorthiamid; cinmethylin; cinosulfuron; clethodim; clodinafop and its ester derivatives (for example clodinafop-propargyl); clomazone; clomeprop; cloproxydim; clopyralid; cumyluron (JC 940); cyanazine; cycloate; cyclosulfamuron (AC 104); cycloxydim; cycluron; cyhalofop and its ester derivatives (for example butylester, DEH-112); cyperquat; cyprazine; cyprazole; daimuron; 2,4-DB; dalapon; desmedipham; desmetryn; di-allate; dicamba; dichlobenil; dichlorprop; diclofop and its esters such as diclofop-methyl; diethatyl; difenoxuron; difenzoquat; diflufenican; dimefuron; dimethachlor; dimethametryn; dimethenamid (SAN-582H); dimethazone, clomazon; dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole-4-carboxamide; endothal; EPTC; esprocarb; ethalfluralin; ethametsulfuron-methyl; ethidimuron; ethiozin; ethofumesate; F5231, i.e. N-[2-chloro-4-fluoro-5-[4-(3-fluoropropyl)-4,5-dihydro-5-oxo-1H-tetrazol-1-yl]phenyl]ethanesulfonamide; ethoxyfen and its esters (for example ethylester, HN-252); etobenzanid (HW 52); fenoprop; fenoxan, fenoxaprop and fenoxaprop-P and their esters, for example fenoxaprop-P-ethyl and fenoxaprop-ethyl; fenoxydim; fenuron; flamprop-methyl; flazasulfuron; fluazifop and fluazifop-P and their esters, for example fluazifop-butyl and fluazifop-P-butyl; fluchloralin; flumetsulam; flumeturon; flumiclorac and its esters (for example pentylester, S-23031); flumioxazin (S482); flumipropyn; flupoxam (KNW-739); fluorodifen; fluoroglycofen-ethyl; flupropacil (UBIC4243); fluridone; flurochloridone; fluroxypyr; flurtamone; fomesafen; fosamine; furyloxyfen; glufosinate; glyphosate; halosafen; halosulfuron and its esters (for example methylester, NC-319); haloxyfop and its esters; haloxyfop-P (=R-haloxyfop) and its esters; hexazinone; imazapyr; imazamethabenz-methyl; imazaquin and salts such as the ammonium salt; ioxynil; imazethamethapyr; imazethapyr; imazosulfuron; isocarbamid; isopropalin; isoproturon; isouron; isoxaben; isoxapyrifop; karbutilate; lactofen; lenacil; linuron; MCPA; MCPB; mecoprop; mefenacet; mefluidid; metamitron; metazachlor; metham; methabenzthiazuron; methazole; methoxyphenone; methyldymron; metabenzuron, methobenzuron; metobromuron; metolachlor; metosulam (XRD 511); metoxuron; metribuzin; metsulfuron-methyl; MH; molinate; monalide; monolinuron; monuron; monocarbamide dihydrogensulfate; MT 128, i.e. 6-chloro-N-(3-chloro-2-propenyl)-5-methyl-N-phenyl-3-pyridazinamine; MT 5950, i.e. N-[3-chloro-4-(1-methylethyl)phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazon; oxyfluorfen; paraquat; pebulate; pendimethalin; perfluidone; phenisopham; phenmedipham; picloram; pinoxaden; piperophos; piributicarb; pirifenop-butyl; pretilachlor; primisulfuron-methyl; procyazine; prodiamine; profluralin; proglinazine-ethyl; prometon; prometryn; propachlor; propanil; propaquizafop and its esters; propazine; propham; propisochlor; propyzamide; prosulfalin; prosulfocarb; prosulfuron (CGA-152005); prynachlor; pyrazolinate; pyraclonil, pyrazon; pyrazosulfuron-ethyl; pyrazoxyfen; pyridate; pyrithiobac (KIH-2031); pyroxofop and its esters (for example propargyl ester); quinclorac; quinmerac; quinofop and its ester derivatives, quizalofop and quizalofop-P and their ester derivatives for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and its methyl ester; sulfentrazon (FMC-97285, F-6285); sulfazuron; sulfometuron-methyl; sulfosate (ICI-A0224); TCA; tebutam (GCP-5544); tebuthiuron; terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiazafluron; thiazopyr (Mon-13200); thidiazimin (SN-24085); thiobencarb; thifensulfuron-methyl; tiocarbazil; tralkoxydim; tri-allate; triasulfuron; triazofenamide; tribenuron-methyl; triclopyr; tridiphane; trietazine; trifluralin; triflusulfuron and esters (for example methyl ester, DPX-66037); trimeturon; tsitodef; vernolate; WL 110547, i.e. 5-phenoxy-1-[3-(trifluoromethyl)phenyl]-1H-tetrazole; UBH-509; D489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127, KIH-2023 and KIH-485.

For use, the formulations, which are present in commercially available form, are if appropriate diluted in the customary manner, for example using water in the case of wettable powders, emulsifiable concentrates, dispersions and water-dispersible granules. Preparations in the form of dusts, soil granules, granules for spreading and sprayable solutions are usually not diluted any further with other inert substances prior to use.

The application rate required of the compounds of the formula (I) varies with the external conditions such as, inter alia, temperature, humidity and the nature of the herbicide used. It can vary within wide limits, for example between 0.001 and 1.0 kg/ha or more of active substance, but it is preferably between 0.005 and 750 g/ha.

The examples which follow illustrate the invention.

### A. Chemical Examples

1. Preparation of 3-fluoro-5-(1-methyl-3-trifluoromethylpyrazol-5-yloxy)nitrobenzene

Under an atmosphere of nitrogen, 30.00 g (189 mmol) of 3,5-difluoronitrobenzene were initially charged in 150 ml of N,N-dimethylformamide, and 28.67 g (207 mmol) of K₂CO₃ and 31.32 g (189 mmol) of 1-methyl-3-(trifluoromethyl)pyrazol-5-one were added at room temperature (RT). The mixture was heated at 85° C. for 33 h and at 100° C. for a further 3 h and then cooled to RT, and water was added to the reaction solution. The mixture was extracted three times with ethyl acetate. The combined phases were washed with water and then dried over MgSO₄, filtered and concentrated. Column chromatography of the crude product gave 9.00 g of 3-fluoro-5-(1-methyl-3-trifluoromethylpyrazol-5-yloxy)nitrobenzene in the form of an orange-red oil.

¹H-NMR: δ[CDCl₃] 3.84 ppm (s, 3H), 6.07 (s, 1H), 7.20 (dt, 1H), 7.80 (m, 2H)

2. Preparation of 3-(1-methyl-5-trifluoromethylpyrazol-3-yloxy)-5-(3-trifluoromethylphenyloxy)nitrobenzene

Under an atmosphere of nitrogen, 3.64 g (22.0 mmol) of 1-methyl-5-(trifluoromethyl)-pyrazol-3-one were initially charged in 100 ml of dimethylacetamide, and 0.598 g (25.0 mmol) of NaH (80% pure) was added at 0° C. The mixture was allowed to warm to RT, 6.00 g (20.0 mmol) of 3-fluoro-5-(3-trifluoromethylphenyloxy)nitrobenzene were added and the mixture was heated at 90° C. for 2 h and at 130° C. for a further 8 h and then cooled to RT, and water was added to the reaction solution, which was then stirred for a number of minutes. The mixture was extracted twice with heptane/ethyl acetate (1:1) and twice with ethyl acetate. The combined phases were washed with water and then dried over MgSO₄, filtered and concentrated. Column chromatography of the crude product gave 4.24 g of 3-(1-methyl-5-trifluoromethylpyrazol-3-yloxy)-5-(3-trifluoromethylphenyloxy)nitrobenzene in the form of wax-like crystals.

¹H-NMR: δ[CDCl₃] 3.92 ppm (s, 3H), 6.28 (s, 1H), 7.14 (t, 1H), 7.24 (dt, 1H), 7.34 (s, 1H), 7.45-7.60 (m, 2H), 7.75 (t, 1H).

3. Preparation of 3-(1-methyl-5-trifluoromethylpyrazol-3-yloxy)-5-(3-trifluoromethylphenyloxy)aniline

Under an atmosphere of nitrogen, 4.00 g (9.0 mmol) of 3-(1-methyl-5-trifluoromethylpyrazol-3-yloxy)-5-(3-trifluoromethylphenyloxy)nitrobenzene, 5.00 g (45.0 mmol) of ammonium formate and 0.50 g (4.0 mmol) of Pd(OH)₂ on carbon (20% pure) were initially charged in 100 ml of methanol and heated at 70° C. for 90 min. The reaction solution was cooled to RT, filtered and concentrated, giving 3.75 g of 3-(1-methyl-5-trifluoromethylpyrazol-3-yloxy)-5-(3-trifluoromethylphenyloxy)aniline as a yellow oil.

¹H-NMR: δ[CDCl₃] 3.88 ppm (s, 3H), 6.08 (t, 1H), 6.15 (t, 1H), 6.17 (s, 1H), 6.23, (t, 1H), 7.20 (d, 1H), 7.26 (s, 1H), 7.34 (d, 1H), 7.43 (t, 1H).

4. Preparation of 1-(1-methyl-5-trifluoromethylpyrazol-3-yloxy)-3-(3-trifluoromethylphenyloxy)benzene

Under an atmosphere of nitrogen, 0.215 g of 3-(1-methyl-5-trifluoromethylpyrazol-3-yloxy)-5-(3-trifluoromethylphenyloxy)aniline and 0.159 g (2.0 mmol) of n-butyl nitrite were initially charged in 5 ml of THF and heated at 40° C. for 34 h. The reaction solution was cooled to RT, water was added and the mixture was stirred for a number of minutes and extracted twice with ethyl acetate. The combined phases were washed with water and then dried over MgSO₄, filtered and concentrated. Column chromatography of the crude product gave 60 mg of 1-(1-methyl-5-trifluoromethylpyrazol-3-yloxy)-3-(3-trifluoromethylphenyloxy)benzene as a yellow oil.

¹H-NMR: δ [CDCl₃] 3.85 ppm (s, 3H), 6.08 (s, 1H), 6.77 (dt, 1H), 6.80 (t, 1H), 6.90, (dt, 1H), 7.20 (dt, 1H), 7.28-7.38 (m, 3H), 7.43 (t, 1H).

5. Preparation of 3-(1-methyl-5-trifluoromethylpyrazol-3-yloxy)-5-(3-trifluoromethylphenyloxy)fluorobenzene

Under protective gas, 0.185 g (2.0 mmol) of nitrosyl tetrafluoroborate was added at 0° C. to 0.220 g of 3-(1-methyl-5-trifluoromethylpyrazol-3-yloxy)-5-(3-trifluoromethylphenyloxy)aniline dissolved in 5 ml of chlorobenzene, and the mixture was stirred at RT for 30 min and then heated at 90° C. for 3 h. The mixture was cooled to RT, water was added and the mixture was stirred for a number of minutes and extracted twice with ethyl acetate. The combined phases were washed with water and then dried over MgSO₄, filtered and concentrated. Column chromatography of the crude product gave 40 mg of 3-(1-methyl-5-trifluoromethylpyrazol-3-yloxy)-5-(3-trifluoromethylphenyloxy)fluorobenzene as a yellow oil.

¹H-NMR: δ [CDCl₃] 3.85 ppm (s, 3H), 6.21 (s, 1H), 6.43 (dt, 1H), 6.58 (s, 1H), 6.63, (dt, 1H), 7.22 (dt, 1H), .7.30 (s, 1H), 7.38-7.50 (bm, 2H).

6. Preparation of 3-(1-methyl-5-trifluoromethylpyrazol-3-yloxy)-5-(1-methyl-3-trifluoromethylpyrazol-5-yloxy)fluorobenzene

Under protective gas, 126 mg of 1-methyl-5-(trifluoromethyl)pyrazol-3-one were initially charged in 5 ml of dimethylacetamide, and 27 mg of NaH (80% pure) were added at 0° C. The mixture was allowed to warm to RT, 200 mg of 5-(1-methyl-3-trifluoromethylpyrazol-5-yloxy)-1,3-difluorobenzene and catalytic amounts of copper iodide were added, the mixture was heated at 150° C. for 6 h and cooled to RT, water was added and the mixture was stirred for a number of minutes. The mixture was extracted three times with ethyl acetate. The combined phases were washed with water, dried over MgSO₄, filtered and concentrated. Column chromatography of the crude product gave 200 mg of 3-(1-methyl-5-trifluoromethylpyrazol-3-yloxy)-5-(1-methyl-3-trifluoromethylpyrazol-5-yloxy)fluorobenzene as a yellow oil.

¹H-NMR: δ[CDCl₃] 3.78 ppm (s, 3H), 3.91 (s, 3H), 6.01 (s, 1H), 6.24 (s, 1H), 6.58 (dt, 1H), 6.70 (m, 2H).

7. Preparation of 3-(1-methyl-5-trifluoromethylpyrazol-3-yloxy)-5-(3-trifluoromethylphenyloxy)chlorobenzene

0.400 g of 3-(1-methyl-5-trifluoromethylpyrazol-3-yloxy)-5-(3-trifluoromethylphenyloxy)aniline was dissolved in 5 ml of methylene chloride, 0.569 g (6.0 mmol) of copper(I) chloride and then 0.593 g (6.0 mmol) of n-butyl nitrite were added at 0-5° C. and the mixture was stirred at 0-5° C. for 2-3 h. The reaction solution was allowed to warm to RT overnight, water was added and the mixture was stirred for a number of minutes and extracted twice with methylene chloride. The combined phases were washed with water and then dried over MgSO₄, filtered and concentrated. Column chromatography of the crude product gave 60 mg of 1-(1-methyl-5-trifluoromethylpyrazol-3-yloxy)-3-(3-trifluoromethylphenyloxy)-chlorobenzene as an orange oil.

¹H-NMR: δ[CDCl₃] 3.84 ppm (s, 3H), 6.18 (s, 1H), 6.80 (m, 2H), 6.91 (dt, 1H), 7.18 (dt, 1H), 7.28-7.50 (m, 3H).

The examples given in the tables which follow were prepared analogously to the abovementioned methods, or can be prepared analogously to the abovementioned methods.

The abbreviations used denote

Et=ethyl Me=methyl

TABLE A
Compounds of the formula (I) according to the invention in which the
substituents and symbols are as defined below:
A = A1 R⁴ = CF₃

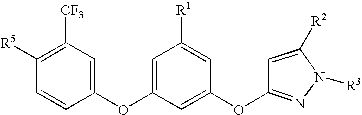

| No. | R¹ | R² | R³ | R⁵ | Physical data: ¹H-NMR (CDCl₃) |
|---|---|---|---|---|---|
| 1 | H | CF₃ | Me | H | 6.18 (s, pyrazolyl-H) |
| 2 | F | CF₃ | Me | H | 6.21 (s, pyrazolyl-H) |
| 3 | Cl | CF₃ | Me | H | 6.21 (s, pyrazolyl-H) |
| 4 | Br | CF₃ | Me | H | 6.21 (s, pyrazolyl-H) |
| 5 | I | CF₃ | Me | H | 6.20 (s, 1H, pyrazolyl-H) |
| 6 | H | CF₃ | Et | H | |
| 7 | F | CF₃ | Et | H | |
| 8 | Cl | CF₃ | Et | H | |
| 9 | Br | CF₃ | Et | H | |
| 10 | I | CF₃ | Et | H | |
| 11 | H | CHF₂ | Me | H | |
| 12 | F | CHF₂ | Me | H | |
| 13 | Cl | CHF₂ | Me | H | |
| 14 | Br | CHF₂ | Me | H | |
| 15 | I | CHF₂ | Me | H | |
| 16 | H | CHF₂ | Et | H | |
| 17 | F | CHF₂ | Et | H | |
| 18 | Cl | CHF₂ | Et | H | |
| 19 | Br | CHF₂ | Et | H | |
| 20 | I | CHF₂ | Et | H | |
| 21 | H | CF₃ | Me | F | 6.19 (s, pyrazolyl-H) |
| 22 | F | CF₃ | Me | F | 6.22 (s, pyrazolyl-H) |
| 23 | Cl | CF₃ | Me | F | 6.21 (s, pyrazolyl-H) |
| 24 | Br | CF₃ | Me | F | 6.21 (s, pyrazolyl-H) |
| 25 | I | CF₃ | Me | F | 6.20 (s, pyrazolyl-H) |
| 26 | H | CF₃ | Et | F | |
| 27 | F | CF₃ | Et | F | |
| 28 | Cl | CF₃ | Et | F | |
| 29 | Br | CF₃ | Et | F | |
| 30 | I | CF₃ | Et | F | |
| 31 | H | CHF₂ | Me | F | |
| 32 | F | CHF₂ | Me | F | |
| 33 | Cl | CHF₂ | Me | F | |
| 34 | Br | CHF₂ | Me | F | |
| 35 | I | CHF₂ | Me | F | |
| 36 | H | CHF₂ | Et | F | |
| 37 | F | CHF₂ | Et | F | |
| 38 | Cl | CHF₂ | Et | F | |
| 39 | Br | CHF₂ | Et | F | |
| 40 | I | CHF₂ | Et | F | |
| 41 | SMe | CF₃ | Me | H | 6.18 |
| 42 | SMe | CF₃ | Me | F | 6.18 |
| 43 | SMe | CF₃ | Et | H | |
| 44 | SMe | CF₃ | Et | F | |
| 45 | SMe | CHF₂ | Me | H | |
| 46 | SMe | CHF₂ | Me | F | |
| 47 | SMe | CHF₂ | Et | H | |
| 48 | SMe | CHF₂ | Et | F | |

TABLE B
Compounds of the formula (I) according to the invention in which the
substituents and symbols are as defined below:
A = A2

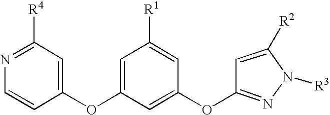

| | | | | | Physical data: ¹H-NMR |
|---|---|---|---|---|---|
| No. | R¹ | R² | R³ | R⁴ | (CDCl₃) |
| 1 | H | CF₃ | Me | OCF₂H | 6.21 (s, pyrazolyl-H) |
| 2 | F | CF₃ | Me | OCF₂H | 6.23 (s, pyrazolyl-H) |
| 3 | Cl | CF₃ | Me | OCF₂H | 6.23 (s, pyrazolyl-H) |
| 4 | Br | CF₃ | Me | OCF₂H | 6.22 (s, pyrazolyl-H) |
| 5 | I | CF₃ | Me | OCF₂H | 6.22 (s, pyrazolyl-H) |
| 6 | H | CF₃ | Et | OCF₂H | |
| 7 | F | CF₃ | Et | OCF₂H | |
| 8 | Cl | CF₃ | Et | OCF₂H | |
| 9 | Br | CF₃ | Et | OCF₂H | |
| 10 | I | CF₃ | Et | OCF₂H | |
| 11 | H | CHF₂ | Me | OCF₂H | |
| 12 | F | CHF₂ | Me | OCF₂H | |
| 13 | Cl | CHF₂ | Me | QCF₂H | |
| 14 | Br | CHF₂ | Me | OCF₂H | |
| 15 | I | CHF₂ | Me | OCF₂H | |
| 16 | H | CHF₂ | Et | OCF₂H | |
| 17 | F | CHF₂ | Et | OCF₂H | |
| 18 | Cl | CHF₂ | Et | OCF₂H | |
| 19 | Br | CHF₂ | Et | OCF₂H | |
| 20 | I | CHF₂ | Et | OCF₂H | |
| 21 | H | CF₃ | Me | Cl | 6.19 (s, pyrazolyl-H) |
| 22 | F | CF₃ | Me | Cl | |
| 23 | Cl | CF₃ | Me | Cl | |
| 24 | Br | CF₃ | Me | Cl | |
| 25 | I | CF₃ | Me | Cl | |
| 26 | H | CF₃ | Et | Cl | |
| 27 | F | CF₃ | Et | Cl | |
| 28 | Cl | CF₃ | Et | Cl | |
| 29 | Br | CF₃ | Et | Cl | |
| 30 | I | CF₃ | Et | Cl | |
| 31 | H | CHF₂ | Me | Cl | |
| 32 | F | CHF₂ | Me | Cl | |
| 33 | Cl | CHF₂ | Me | Cl | |
| 34 | Br | CHF₂ | Me | Cl | |
| 35 | I | CHF₂ | Me | Cl | |
| 36 | H | CHF₂ | Et | Cl | |
| 37 | F | CHF₂ | Et | Cl | |
| 38 | Cl | CHF₂ | Et | Cl | |
| 39 | Br | CHF₂ | Et | Cl | |
| 40 | I | CHF₂ | Et | Cl | |
| 41 | H | CF₃ | Me | CF₃ | 6.22 (s, pyrazolyl-H) |
| 42 | F | CF₃ | Me | CF₃ | 6.23 (s, pyrazolyl-H) |
| 43 | Cl | CF₃ | Me | CF₃ | 6.24 (s, pyrazolyl-H) |
| 44 | Br | CF₃ | Me | CF₃ | 6.23 (s, pyrazolyl-H) |
| 45 | I | CF₃ | Me | CF₃ | |
| 46 | H | CF₃ | Et | CF₃ | |
| 47 | F | CF₃ | Et | CF₃ | |
| 48 | Cl | CF₃ | Et | CF₃ | |
| 49 | Br | CF₃ | Et | CF₃ | |
| 50 | I | CF₃ | Et | CF₃ | |
| 51 | H | CHF₂ | Me | CF₃ | |
| 52 | F | CHF₂ | Me | CF₃ | |
| 53 | Cl | CHF₂ | Me | CF₃ | |
| 54 | Br | CHF₂ | Me | CF₃ | |
| 55 | I | CHF₂ | Me | CF₃ | |
| 56 | H | CHF₂ | Et | CF₃ | |
| 57 | F | CHF₂ | Et | CF₃ | |
| 58 | Cl | CHF₂ | Et | CF₃ | |
| 59 | Br | CHF₂ | Et | CF₃ | |
| 60 | I | CHF₂ | Et | CF₃ | |
| 61 | H | CF₃ | Me | CF₂H | |
| 62 | F | CF₃ | Me | CF₂H | |
| 63 | Cl | CF₃ | Me | CF₂H | |
| 64 | Br | CF₃ | Me | CF₂H | |
| 65 | I | CF₃ | Me | CF₂H | |
| 66 | H | CF₃ | Et | CF₂H | |
| 67 | F | CF₃ | Et | CF₂H | |
| 68 | Cl | CF₃ | Et | CF₂H | |
| 69 | Br | CF₃ | Et | CF₂H | |
| 70 | I | CF₃ | Et | CF₂H | |
| 71 | H | CHF₂ | Me | CF₂H | |
| 72 | F | CHF₂ | Me | CF₂H | |
| 73 | Cl | CHF₂ | Me | CF₂H | |
| 74 | Br | CHF₂ | Me | CF₂H | |
| 75 | I | CHF₂ | Me | CF₂H | |
| 76 | H | CHF₂ | Et | CF₂H | |
| 77 | F | CHF₂ | Et | CF₂H | |
| 78 | Cl | CHF₂ | Et | CF₂H | |
| 79 | Br | CHF₂ | Et | CF₂H | |
| 80 | I | CHF₂ | Et | CF₂H | |
| 81 | SMe | CF₃ | Me | Cl | |
| 82 | SMe | CF₃ | Me | OCF₂H | |
| 83 | SMe | CF₃ | Me | CF₃ | |
| 84 | SMe | CF₃ | Me | CF₂H | |
| 85 | SMe | CF₃ | Et | Cl | |
| 86 | SMe | CF₃ | Et | OCF₂H | |
| 87 | SMe | CF₃ | Et | CF₃ | |
| 88 | SMe | CF₃ | Et | CF₂H | |
| 89 | SMe | CHF₂ | Me | Cl | |
| 90 | SMe | CHF₂ | Me | OCF₂H | |
| 91 | SMe | CHF₂ | Me | CF₃ | |
| 92 | SMe | CHF₂ | Me | CF₂H | |
| 93 | SMe | CHF₂ | Et | Cl | |
| 94 | SMe | CHF₂ | Et | OCF₂H | |
| 95 | SMe | CHF₂ | Et | CF₃ | |
| 96 | SMe | CHF₂ | Et | CF₂H | |

TABLE C
Compounds of the formula (I) according to the invention in which the
substituents and symbols are as defined below:
A = A3 R⁴ = CF₃

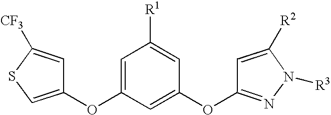

| No. | R¹ | R² | R³ | Physical data: ¹H-NMR (CDCl₃) |
|---|---|---|---|---|
| 1 | H | CF₃ | Me | |
| 2 | F | CF₃ | Me | |
| 3 | Cl | CF₃ | Me | |
| 4 | Br | CF₃ | Me | |
| 5 | I | CF₃ | Me | |
| 6 | H | CF₃ | Et | |
| 7 | F | CF₃ | Et | |
| 8 | Cl | CF₃ | Et | |
| 9 | Br | CF₃ | Et | |
| 10 | I | CF₃ | Et | |
| 11 | H | CHF₂ | Me | |
| 12 | F | CHF₂ | Me | |
| 13 | Cl | CHF₂ | Me | |
| 14 | Br | CHF₂ | Me | |
| 15 | I | CHF₂ | Me | |
| 16 | H | CHF₂ | Et | |
| 17 | F | CHF₂ | Et | |
| 18 | Cl | CHF₂ | Et | |
| 19 | Br | CHF₂ | Et | |
| 20 | I | CHF₂ | Et | |
| 21 | SMe | CF₃ | Me | |
| 22 | SMe | CF₃ | Et | |
| 23 | SMe | CHF₂ | Me | |
| 24 | SMe | CHF₂ | Et | |

TABLE D
Compounds of the formula (I) according to the invention in which the
substituents and symbols are as defined below:
A = A4 R⁶ = Me

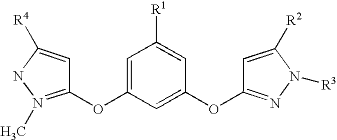

| No. | R¹ | R² | R³ | R⁴ | Physical data: ¹H-NMR (CDCl₃) |
|---|---|---|---|---|---|
| 1 | H | CF₃ | Me | CF₃ | 5.93 and 6.21, each |
| | | | | | (s, pyrazolyl-H) |
| 2 | F | CF₃ | Me | CF₃ | 6.01 and 6.24, each |
| | | | | | (s, pyrazolyl-H) |
| 3 | Cl | CF₃ | Me | CF₃ | 5.99 and 6.23, each |
| | | | | | (s, pyrazolyl-H) |
| 4 | Br | CF₃ | Me | CF₃ | 5.98 and 6.22, each |
| | | | | | (s, pyrazolyl-H) |
| 5 | I | CF₃ | Me | CF₃ | 5.97 and 6.22, each |
| | | | | | (s, pyrazolyl-H) |
| 6 | H | CF₃ | Et | CF₃ | |
| 7 | F | CF₃ | Et | CF₃ | 6.03 and 6.22, each |
| | | | | | (s, pyrazolyl-H) |
| 8 | Cl | CF₃ | Et | CF₃ | |
| 9 | Br | CF₃ | Et | CF₃ | |
| 10 | I | CF₃ | Et | CF₃ | |
| 11 | H | CHF₂ | Me | CF₃ | |
| 12 | F | CHF₂ | Me | CF₃ | |
| 13 | Cl | CHF₂ | Me | CF₃ | |
| 14 | Br | CHF₂ | Me | CF₃ | |
| 15 | I | CHF₂ | Me | CF₃ | |
| 16 | H | CHF₂ | Et | CF₃ | |
| 17 | F | CHF₂ | Et | CF₃ | |
| 18 | Cl | CHF₂ | Et | CF₃ | |
| 19 | Br | CHF₂ | Et | CF₃ | |
| 20 | I | CHF₂ | Et | CF₃ | |
| 21 | H | CF₃ | Me | CHF₂ | |
| 22 | F | CF₃ | Me | CHF₂ | |
| 23 | Cl | CF₃ | Me | CHF₂ | |
| 24 | Br | CF₃ | Me | CHF₂ | |
| 25 | I | CF₃ | Me | CHF₂ | |
| 26 | H | CF₃ | Et | CHF₂ | |
| 27 | F | CF₃ | Et | CHF₂ | |
| 28 | Cl | CF₃ | Et | CHF₂ | |
| 29 | Br | CF₃ | Et | CHF₂ | |
| 30 | I | CF₃ | Et | CHF₂ | |
| 31 | H | CHF₂ | Me | CHF₂ | |
| 32 | F | CHF₂ | Me | CHF₂ | |
| 33 | Cl | CHF₂ | Me | CHF₂ | |
| 34 | Br | CHF₂ | Me | CHF₂ | |
| 35 | I | CHF₂ | Me | CHF₂ | |
| 36 | H | CHF₂ | Et | CHF₂ | |
| 37 | F | CHF₂ | Et | CHF₂ | |
| 38 | Cl | CHF₂ | Et | CHF₂ | |
| 39 | Br | CHF₂ | Et | CHF₂ | |
| 40 | I | CHF₂ | Et | CHF₂ | |
| 41 | SMe | CF₃ | Me | CHF₂ | |
| 42 | SMe | CF₃ | Me | CF₃ | 5.94 and 6.20 |
| 43 | SMe | CF₃ | Et | CHF₂ | |
| 44 | SMe | CF₃ | Et | CF₃ | |
| 45 | SMe | CHF₂ | Me | CHF₂ | |
| 46 | SMe | CHF₂ | Me | CF₃ | |
| 47 | SMe | CHF₂ | Et | CHF₂ | |
| 48 | SMe | CHF₂ | Et | CF₃ | |

### B. Formulation Examples

1. Dust

A dust is obtained by mixing 10 parts by weight of a compound of the formula (I) and 90 parts by weight of talc as inert substance and comminuting the mixture in a hammer mill.

2. Dispersible Powder

A wettable powder which is readily dispersible in water is obtained by mixing 25 parts by weight of a compound of the formula (I), 64 parts by weight of kaolin-containing quartz as inert material, 10 parts by weight of potassium ligninsulfonate and 1 part by weight of sodium oleoylmethyltauride as wetter and dispersant, and grinding the mixture in a pinned-disk mill.

3. Dispersion Concentrate

A dispersion concentrate which is readily dispersible in water is obtained by mixing 20 parts by weight of a compound of the formula (I), 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.), and grinding the mixture in a ball mill to a fineness of below 5 microns.

4. Emulsifiable Concentrate

An emulsifiable concentrate is obtained from 15 parts by weight of a compound of the formula (1), 75 parts by weight of cyclohexanone as solvent and 10 parts by weight of oxethylated nonylphenol as emulsifier.

5. Water-Dispersible Granules

Water-dispersible granules are obtained by mixing

75 parts by weight of a compound of the formula (I),

| | | |
|---|---|---|
| 10 | ″ | calcium lignosulfonate, |
| 5 | ″ | sodium lauryl sulfate, |
| 3 | ″ | polyvinyl alcohol and |
| 7 | ″ | kaolin, |

grinding the mixture in a pinned-disk mill and granulating the powder in a fluidized bed by spraying on water as granulation liquid.

Water-dispersible granules are also obtained by homogenizing and precomminuting, in a colloid mill,

25 parts by weight of a compound of the formula (I),

| | | |
|---|---|---|
| 5 | ″ | sodium 2,2′-dinaphthylmethane-6,6′-disulfonate, |
| 2 | ″ | sodium oleoylmethyltauride, |
| 1 | ″ | polyvinyl alcohol, |
| 17 | ″ | calcium carbonate and |
| 50 | ″ | water, |

subsequently grinding the mixture in a bead mill, and atomizing and drying the resulting suspension in a spray tower by means of a single-substance nozzle.

### C. Biological Examples

1. Pre-Emergence Herbicidal Action Against Harmful Plants

Seeds of mono- and dicotyledonous weeds are placed in sandy loam in cardboard pots and covered with soil. The compounds, which are formulated as wettable powders or emulsion concentrates, are then applied to the surface of the covering soil as aqueous suspension or emulsion at an application rate of 600 to 800 l of water/ha (converted), in a dosage of 1000 g per hectare (converted). Following treatment, the pots are placed in the greenhouse and maintained under good growth conditions for the weeds. The visual scoring of the plant damage or emergence damage is made when the test plants have emerged, after an experimental period of 3 to 4 weeks, in comparison to untreated controls. After the test plants have been left to stand in the greenhouse for 3 to 4 weeks under optimal growth conditions, the effect of the compounds is scored in comparison to compounds disclosed in the prior art. As shown by the results in comparison table 1, the selected compounds according to the invention have better herbicidal activity against a broad spectrum of economically important mono- and dicotyledonous harmful plants than the compounds disclosed in the prior art.

2. Post-Emergence Herbicidal Action Against Harmful Plants

Seeds of mono- and dicotyledonous harmful plants are placed in sandy loam in cardboard pots, covered with soil and grown in the greenhouse under good growth conditions. 2 to 3 weeks after sowing, the test plants are treated in the three-leaf stage. The compounds according to the invention, which are formulated as wettable powders or as emulsion concentrates, are sprayed at an application rate of 600 to 800 l of water/ha (converted) in a dosage of 1000 g per hectare (converted) onto the surface of the green plant parts. After the test plants have been left to stand in the greenhouse for 3 to 4 weeks under optimal growth conditions, the action of the compounds is scored in comparison to compounds disclosed in the prior art. As the results of the comparison tables 2 to 4 show, the selected compounds according to the invention have better herbicidal activity against a broad spectrum of economically important monocotyledonous and dicotyledonous harmful plants than the compounds disclosed in the prior art.

3. Crop Plant Tolerance

In further greenhouse experiments, seeds of barley and of monocotyledonous and dicotyledonous harmful plants are placed in sandy loam, covered with soil and placed in the greenhouse until the plants have developed two to three true leaves. Then, they are treated with the compounds of the formula (I) according to the invention and, in comparison, with those disclosed in the prior art as described above under item 1. Four to five weeks after the application and after having been left to stand in the greenhouse, visual scoring reveals that the compounds according to the invention leave the crop plant undamaged even at relatively high dosages of active compound, in contrast to the compounds disclosed in the prior art.

Compounds According to the Invention

| No. | Structure |
|---|---|
| E1 | 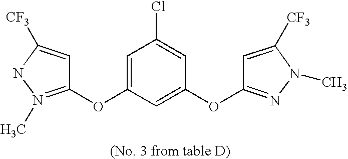 |
| E3 | 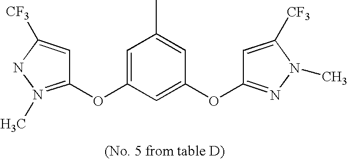 |
| E4 | 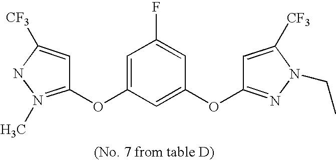 |

Compounds Known from the Prior Art (WO 2003/051846):

| No. | Structure |
|---|---|
| S1 | 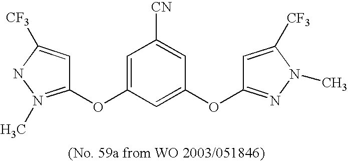 |
| S2 | 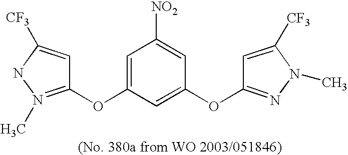 |

The abbreviations used in the comparison tables below have the following meanings:

AMARE Amaranthus retroflexus AVESA Avena fatua

LOLMU Lolium multiflorum SETVI Setaria viridis

SINAL Sinapis arvensis STEME Stellaria media

Comparison Table 1, Pre-Emergence

| | Dosage | Damage of the harmful plants in % | | |
|---|---|---|---|---|
| Compound No. | [g of a.i./ha] | AVESA | SINAL | STEME |
| E1 | 1000 | 90% | 100% | 100% |
| E4 | 1000 | 100% | 100% | 100% |
| S2 | 1000 | 70% | 70% | 90% |

Comparison Table 2, Post-Emergence

| | Dosage | Damage of the harmful plants in % | | | | |
|---|---|---|---|---|---|---|
| Compound No. | [g of a.i./ha] | AMARE | AVESA | LOLMU | SETVI | SINAL |
| E1 | 1000 | 100% | 90% | 100% | 90% | 90% |
| S1 | 1000 | 80% | 80% | 80% | 80% | 80% |

Comparison Table 3, Post-Emergence

| | Dosage | Damage of the harmful plants in % | | | |
|---|---|---|---|---|---|
| Compound No. | [g of a.i./ha] | AVESA | LOLMU | SETVI | STEME |
| E3 | 1000 | 80% | 90% | 90% | 90% |
| S2 | 1000 | 60% | 60% | 70% | 80% |

Comparison Table 4, Post-Emergence

| | Dosage | Damage of the harmful plants in % | | | |
|---|---|---|---|---|---|
| Compound No. | [g of a.i./ha] | AMARE | SETVI | SINAL | STEME |
| E4 | 1000 | 100% | 100% | 100% | 100% |
| S1 | 1000 | 80% | 80% | 80% | 80% |
| S2 | 1000 | 70% | 90% | 90% | 80% |

1. A pyrazolyloxyphenyl derivative of the formula (I) or a salt thereof 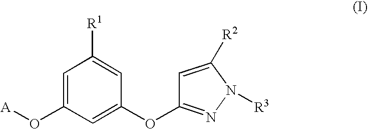
in which the substituents and indices are as defined below:
R¹ is hydrogen, bromine, chlorine, fluorine, iodine, or methylthio;
R² is trifluoromethyl, difluoromethyl or chlorodifluoromethyl;
R³ is methyl or ethyl;
A is a radical from the group consisting of the radicals A1 to A4 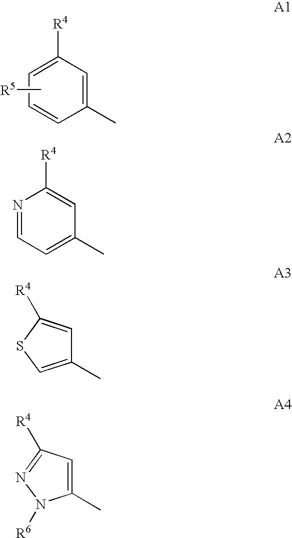
R⁴ is fluoromethyl, difluoromethyl, trifluoromethyl, fluoromethoxy, difluoromethoxy, trifluoromethoxy, chlorine or cyano;
R⁵ is hydrogen, (C₁-C₈)-alkyl, bromine, chlorine, fluorine, iodine or cyano, and
R⁶ is (C₁-C₈)-alkyl.

2. The pyrazolyloxyphenyl derivative as claimed in claim 1, in which
R¹ is hydrogen, bromine, chlorine, fluorine, iodine or methylthio;
R² is trifluoromethyl or difluoromethyl;
R³ is methyl or ethyl;
A is a radical from the group consisting of the radicals A1 to A4;
R⁴ is difluoromethyl, trifluoromethyl, difluoromethoxy, trifluoromethoxy, chlorine or cyano;
R⁵ is hydrogen, fluorine or chorine;
R⁶ is methyl or ethyl.

3. The pyrazolyloxyphenyl derivative as claimed in claim 1, in which
R¹ is hydrogen, bromine, chlorine, fluorine, iodine or methylthio;
R² is trifluoromethyl or difluoromethyl;
R³ is methyl or ethyl;
A is a radical from the group consisting of the radicals A1 to A4;
R⁴ is difluoromethyl, trifluoromethyl, difluoromethoxy, trifluoromethoxy, chlorine or cyano;
R⁵ is hydrogen or fluorine;
R⁶ is methyl.

4. The pyrazolyloxyphenyl derivative as claimed in claim 1, in which
R¹ is hydrogen, bromine, chlorine, fluorine or iodine;
R² is trifluoromethyl or difluoromethyl;
R³ is methyl or ethyl;
A is the radical A1;
R⁴ is difluoromethyl, trifluoromethyl, difluoromethoxy, trifluoromethoxy, chlorine or cyano;
R⁵ is hydrogen or fluorine.

5. The pyrazolyloxyphenyl derivative as claimed in claim 1, in which
R¹ is hydrogen, bromine, chlorine, fluorine or iodine;
R² is trifluoromethyl or difluoromethyl;
R³ is methyl or ethyl;
A is the radical A2;
R⁴ is difluoromethyl, trifluoromethyl, difluoromethoxy, trifluoromethoxy, chlorine or cyano.

6. The pyrazolyloxyphenyl derivative as claimed in claim 1, in which
R¹ is hydrogen, bromine, chlorine, fluorine or iodine;
R² is trifluoromethyl or difluoromethyl;
R³ is methyl or ethyl;
A is the radical A3;
R⁴ is difluoromethyl, trifluoromethyl, difluoromethoxy, trifluoromethoxy, chlorine or cyano.

7. The pyrazolyloxyphenyl derivative as claimed in claim 1, in which
R¹ is hydrogen, bromine, chlorine, fluorine or iodine;
R² is trifluoromethyl or difluoromethyl;
R³ is methyl or ethyl;
A is the radical A4;
R⁴ is difluoromethyl, trifluoromethyl, difluoromethoxy, trifluoromethoxy, chlorine or cyano;
R⁶ represents methyl.

8. A herbicidal composition which comprises a herbicidally effective amount of at least one compound of the formula (I) as claimed in claim 1.

9. The herbicidal composition as claimed in claim 8 in a mixture with formulation auxiliaries.

10. A method for controlling unwanted plants which comprises applying an effective amount of at least one compound of the formula (I) as claimed in claim 1 to the plants or the location of the unwanted vegetation.

11. (canceled)

12. (canceled)

13. (canceled)

14. A method for controlling unwanted plants which comprises applying an effective amount of a herbicidal composition as claimed in claim 8 to the plants or the location of the unwanted vegetation.

15. The method of claim 10, wherein the unwanted plants are present in crops of useful plants.

16. The method of claim 15, wherein the useful plants are transgenic useful plants.

17. The method of claim 14, wherein the unwanted plants are present in crops of useful plants.

18. The method of claim 17, wherein the useful plants are transgenic useful plants.
